(19) United States
(12) Patent Application Publication
(10) Pub. No.: US 2012/0016218 A1
(43) Pub. Date: Jan. 19, 2012

(54) DISCRIMINATION OF CHEYNE-STOKES BREATHING PATTERNS BY USE OF OXIMETRY SIGNALS

(75) Inventors: Chun Yui Lau, New South Wales (AU); Jeffrey Peter Armitstead, New South Wales (AU)

(73) Assignee: RESMED LIMITED, Bella Vista, NSW (AU)

(21) Appl. No.: 13/259,649

(22) PCT Filed: Apr. 15, 2010
(86) PCT No.: PCT/AU10/00416

§ 371 Date: Sep. 23, 2011

Related U.S. Application Data
(60) Provisional application No. 61/170,734, filed on Apr. 20, 2009.

(51) Int. Cl. A61B 5/1455

(52) U.S. Cl. 600/323

(57) ABSTRACT

Methods and apparatus provide Cheyne-Stokes respiration (“CSR”) detection based on a blood gas measurements such as oximetry. In some embodiments, a duration, such as a mean duration of contiguous periods of changing saturation or re-saturation occurring in an epoch taken from a processed oximetry signal, is determined. An occurrence of CSR may be detected from a comparison of the duration and a threshold derived to differentiate saturation changes due to CSR respiration and saturation changes due to obstructive sleep apnea. The threshold may be a discriminant function derived as a classifier by an automated training method. The discriminant function may be further implemented to characterize the epoch for CSR based on a frequency analysis of the oximetry data. Distance from the discriminant function may be utilized to generate probability values for the CSR detection.

### CROSS-REFERENCE TO RELATED APPLICATIONS

This application claims the benefit of the filing date of U.S. Provisional Patent Application No. 61/170,734, filed Apr. 20, 2009, the disclosure of which is hereby incorporated herein by reference.

### FIELD OF THE TECHNOLOGY

This technology relates to the discrimination of breathing abnormalities by applying quantitative measures to a physiological signal for use as a clinical decision-support tool. In particular it relates to the discrimination of Cheyne-Stokes Respiration (“CSR”) by the analysis of oximetry signals, which may optionally be in conjunction with flow signals. The technology may also relate to the training of a classifier able to provide probability values for CSR discrimination. The technology may also relate to techniques for improving the readout of oximetry signals by removing artifacts recognizable in the context of CSR.

### BACKGROUND OF THE TECHNOLOGY

The diagnosis of CSR usually involves conducting a sleep study and analyzing the resulting polysomnography (“PSG”) data. In a full diagnostic PSG study, a range of biological parameters are monitored that typically include a nasal flow signal, measures of respiratory effort, pulse oximetry, sleeping position, and may include: electroencephalography (“EEG”), electrocardiography (“ECG”), electromyography (“EMG”) and electro-oculography (“EOG”). Breathing characteristics are also identified from visual features, thus allowing a clinician to assess respiratory function during sleep and evaluate any presence of CSR.

During a period of Cheyne-Stokes breathing or CSR, patterns of waxing and waning tidal volume can be seen in a nasal flow signal, which is a direct measure of pulmonary functions. This unstable behavior of breathing often extends its presence into other cardio-respiratory parameters such as blood oxygen saturation levels where cyclical changes can be seen.

While the examination by a clinician is the most comprehensive method, it is a costly process and depends heavily upon clinical experience and understanding. For effective and efficient screening of patients, a classifier algorithm has been developed by the assignee of this invention that automates the scoring process by calculating the probability of a CSR occurring based on a nasal flow signal. The algorithm is disclosed in U.S. patent application Ser. No. 11/576,210 (U.S. Patent App. Pub. No. 20080177195) filed Mar. 28, 2007, and published as WO2006066337A1 Jun. 29, 2006. The existing algorithm is a flow-based classifier where a probability of CSR is calculated given a sequence of discrete flow values. A series of pre-processing steps are performed such as linearization of flow values, filtering and the extraction of respiratory events.

The concept of a classifier is common to many fields where it is desirable to assign an object or an underlying state of an object to one of a number of classes. This concept is used, for example, in the fields of voice recognition (where sound bytes are classified as different words or syllables), radar detection (where visual signals are classified as enemy/friendly targets) and medical diagnosis (where test results are used to classify a patient's disease state). The design of a classifier falls under the field of Pattern Recognition and a classifier can be of the supervised type (the classifier is built from training data which has been pre-classed by a supervisor or “expert”) or unsupervised type (where the natural ordering or clustering of the data determines the different classes). Time signal classification usually relies on representing the signal at particular time points with “features”. Features are simply numbers that distil the essence of the signal at a point in time, a form of compression. A set (or vector) of features is called a “pattern”. A classifier takes a pattern and manipulates it mathematically with a suitable algorithm to produce a probability value for each of a number of classes. The pattern is assigned to the class with the highest probability.

Home pulse oximetry has been proposed as an alternative tool for identification of CSR, but relies on visual inspection of the oximetry signal by a trained observer (Staniforth et al., 1998, Heart, 79:394-99).

A study of 104 subjects with Congestive Heart Failure (“CHF”) by Staniforth et al. (1998, Heart, 79, 394-399.) has examined the de-saturation index recorded in nocturnal oximetry compared to normal controls. The model yielded a specificity of 81% and a sensitivity of 87% for detecting CSR-CSA. However, the overall accuracy of the model was not provided. Those authors made no attempt to determine if pulse oximetry could be used to distinguish between CSR-CSA and Obstructive Sleep Apnea (‘OSA’). U.S. Pat. No. 5,575,285—Takanashi et al, describes measuring oxygen saturation in blood from scattered and transmitted light and performing Fourier transformation to obtain a power spectrum over a frequency range of 500 Hz to 20 kHz. However, that described method does not allow for distinction between patients with CSR and OSA.

U.S. Pat. No. 6,839,581 to Grant et al, PCT Application No. WO 01/076459 and U.S. Published Patent Application No. 2002/0002327 are entitled “Method for Detecting Cheyne-Stokes Respiration in Patients with Congestive Heart Failure.” They jointly propose a diagnostic method for CSR including performing overnight oximetry recordings and performing spectral analysis on the oximetry recordings. A classification tree or neural network based on parameters derived from a power spectral analysis determines the presence or absence of CSR.

U.S. Pat. No 6,760,608 to Lynn is entitled “Oximetry System for Detecting Ventilation Instability.” This patent describes a pulse oximetry system used to generate a time series of oxygen saturation values. The occurrence of certain patterns along the time series is used to indicate ventilation instability.

U.S. Pat. No. 7,081,095 to Lynn et al is entitled “Centralized Hospital Monitoring System for Automatically Detecting Upper Airway Instability and for Preventing and Aborting Adverse Drug Reaction”. It proposes an automatic system of diagnosis of OSA in a computerized environment of a centralized hospital critical care system.

U.S. Pat. No. 7,309,314 to Grant et al is entitled “Method for Predicting Apnea-Hypopnea Index From Overnight Pulse Oximetry Readings.” This patent proposes a tool for predicting an Apopnea Hypopnea Index (“AHI”) for use in the diagnosis of OSA by recording pulse oximetry readings, and obtaining a delta index, oxygen saturation times and oximetry de-saturation events. A multivariate non-parametric analysis and bootstrap aggregation is performed.

U.S. Pat. No. 7,398,115 to Lynn is entitled “Pulse Oximetry Relational Alarm System for Early Recognition of Instability and Catastrophic Occurrences.” The system described in this patent has an alarm triggered by the early recognition of likely catastrophic occurrences by detecting decreases in O₂ saturation coupled with either: a) decrease in pulse rate; or b) increase in respiration rate. The system of this patent is aimed at treating and detecting OSA.

None of these prior art systems are capable of reliably interpreting oximetric data to reliably discriminate OSAs from CSR and to develop a probabilistic value for such attempts at apnea discriminations.

### SUMMARY OF THE TECHNOLOGY

The present technology enhances the discrimination of CSR based on oximetry. The technology may be applied to enhance the detection performance of a flow-based classifier technology system. Thus, it may enable the screening of CSR to become more accessible. For example, it may be implemented as an additional feature to the detection system described in U.S. patent application Ser. No. 11/576,210 filed Mar. 28, 2007, and published as WO 06066337A1 on Jun. 29, 2006. Optionally, the technology may also serve independently or as a stand-alone alternative when a flow signal or data therefrom is unavailable or of unfavorable quality.

The present technology may replace the current screening process with one that is generally more comfortable and easier to use for the patient, easier to administer for the physician and/or less costly to conduct the analysis.

While the present technology may be explained in terms of a sequential process or algorithm, it may be understood that the process or algorithm can be carried out using a non-linear, non-sequential, or non-staged process, or the order of the process may be changed. While this embodiment of the technology describes an entire process, aspects of the technology may relate to only a subset of that process.

A signal representative of respiration, such as an oximetry signal, may be recorded from a patient using a logging device which includes a data-acquisition system and a memory. The respiratory signal may be processed either on-board by the recording device or off-line using a computer.

The signal may be initially pre-processed. For example, the signal can be filtered to remove unwanted noise and, where appropriate, the baseline is zeroed. The signal may also be made linear depending on the transducer used to detect the respiration. In particular the technology may include a process for removing artifacts peculiar to oximetric measurements and for developing an oximetry signal quality indicator (QI) that may be used to determine a confidence level in the discrimination prediction.

In another stage the signal is divided into n epochs of equal length. The epoch length can be as long as the entire record or as short as is practicable to enable detection of respiratory patterns. In one example embodiment, the epoch length is 30 minutes.

A CSR-detection algorithm of the present technology alternatively or in conjunction with oximetry may use the nasal flow signal from a device such as MAP's microMesam® together with pattern recognition techniques to assign a probability of CS breathing to each 30 minute epoch of flow recorded.

The technology may provide a method for the calculation of an Event Feature. The method may also include the calculation of a Spectral Feature determined by, for example, Fourier analysis or by the use of Wavelet Transforms.

Another characteristic of CSR, namely saturation delay, may be used to provide a method for calculating the amount of delay of de-saturation and re-saturation delayed but in synchrony with breathing as a further indicator of CSR.

The technology also may involve a method for training a processor implemented classifier to discriminate CSR and for producing a probability value for each epoch segment of oximetric data for indicating the presence of CSR.

In some embodiments of the technology, a computer implemented method detects an occurrence of Cheyne-Stokes respiration with one or more programmed processors. The method of the processor may include accessing blood gas data representing a measured blood gas signal. It may also include determining a duration of one or more contiguous periods of changing saturation of a blood gas from the blood gas data. It may further include detecting the occurrence of Cheyne-Stokes respiration from a comparison of the determined duration and a threshold derived to differentiate saturation changes due to Cheyne-Stokes respiration and saturation changes due to obstructive sleep apnea. In some embodiments, the one or more contiguous periods of changing saturation may be re-saturation periods and the measured blood gas signal may be an oximetry signal. In still further embodiments, the determined duration may be a mean period length and the detecting may indicate an occurrence when the mean period length exceeds the threshold. In some embodiments, the threshold comprises a discriminant function. The detecting the occurrence may optionally involve determining a distance from the threshold and comparing the distance to a further threshold. The method may also optionally involve determining a presence of a peak in a predetermined frequency range for desaturation and resaturation cycles of the blood gas data and comparing the determined presence to the discriminant function.

Embodiments of the technology may also involve an apparatus to detect an occurrence of Cheyne-Stokes breathing. The apparatus may include a memory for blood gas data representing a measured blood gas signal. The apparatus may also include a processor coupled with the memory. The processor may be configured (a) to determine a duration of one or more contiguous periods of changing saturation of a blood gas from the blood gas data and (b) to detect an occurrence of Cheyne-Stokes respiration from a comparison of the determined duration and a threshold derived to differentiate saturation changes due to Cheyne-Stokes respiration and saturation changes due to obstructive sleep apnea. In some embodiments of this apparatus, the one or more contiguous periods of changing saturation may be re-saturation periods when the measured blood gas signal is an oximetry signal, which may be measured by an optional oximeter. In some embodiments, this determined duration may be a mean period length and the detecting may indicate an occurrence when the mean period length exceeds the threshold, which may optionally be a discriminant function. In processor apparatus may also be configured to detect the occurrence by further determining a distance from the discriminant function and comparing the distance to a further threshold. In still further embodiments, the processor can be configured to determine a presence of a peak in a predetermined frequency range for de-saturation and re-saturation cycles of the blood gas data and then compare the determined presence to the discriminant function.

Other features of the technology will be apparent from consideration of the information contained in the following detailed description, abstract and claims.

### BRIEF DESCRIPTION OF THE DRAWINGS

The present technology is illustrated by way of example, and not by way of limitation, in the figures of the accompanying drawings, in which like reference numerals refer to similar elements including:

FIG. 1 is an example graph of the amplitude and first difference of an oximetry signal in a patient over a duration of one half hour (1800 seconds);

FIG. 2 shows the mean saturation duration in CSR as a function of time measured in seconds;

FIG. 3 shows the mean saturation duration in OSA as a function of time measured in seconds;

FIG. 4 shows the spectral feature of CSR, where the spectral feature is the difference between the maximum and mean value of the Fourier Transform of the saturation;

FIG. 5 shows the spectral feature of OSA, where the spectral feature is the difference between the maximum and mean value of the Fourier Transform of the saturation;

FIG. 6 shows the oxygen saturation of representative CSR epochs;

FIG. 7 shows the global wavelet spectrum of CSR as a function of the Fourier-equivalent frequency;

FIG. 8 shows the computed delay for oxygen saturation, ventilation and delayed ventilation as a function of time in seconds;

FIG. 9 depicts a decision boundary and its relationship to the distribution of the training set of data;

FIGS. 10 and 11 depicts the decision boundary and its relationship to the distribution of the validation set of data;

FIG. 12 is an example flow chart for process steps involved in modifying the distribution of data or classifying oximetry epochs for CSR;

FIG. 13 shows schematically the use of the classifier of the present technology to screen patients for evidence of CSR as a computer-aided diagnostic tool;

FIG. 14 shows receiver operating characteristics on a patient-by-patient basis;

FIG. 15 is a further illustration of components of a CSR detection and/or training system of some embodiments of the present technology.

### DETAILED DESCRIPTION

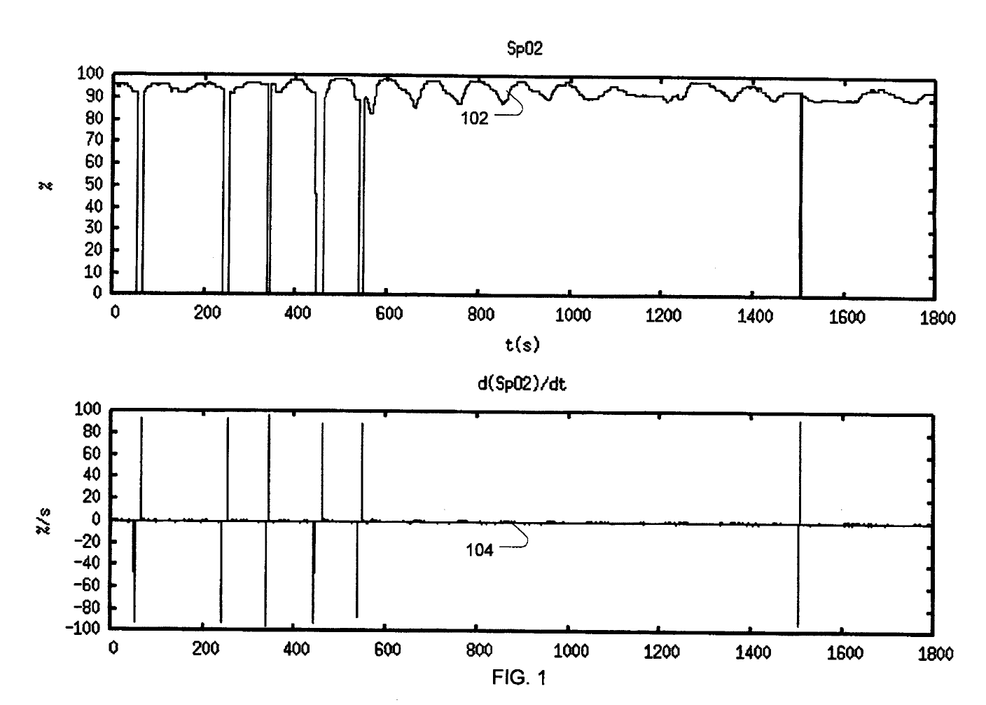

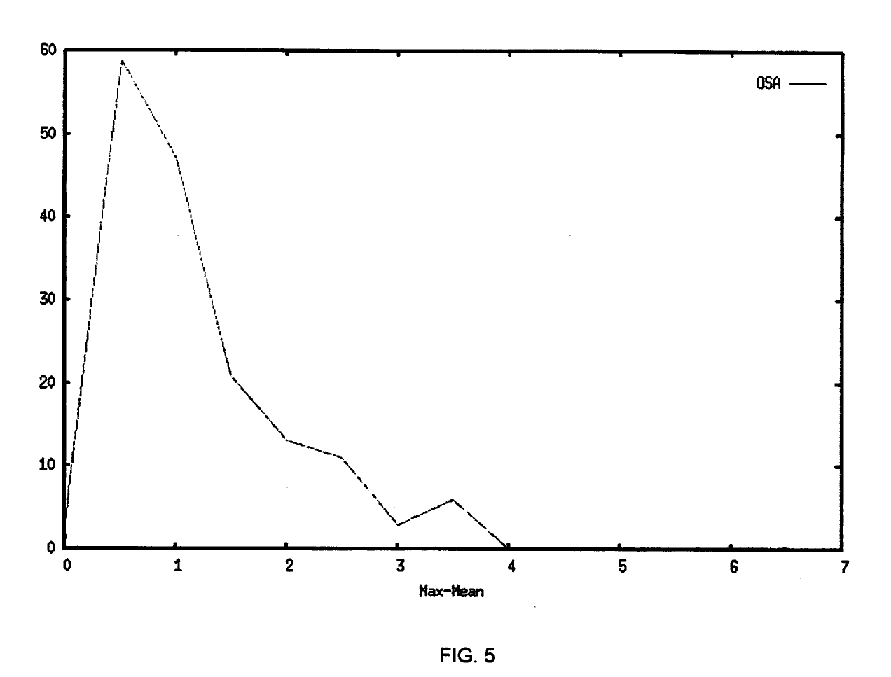

Embodiments of the present technology may include: a system, device, classifier, and/or methods. Example embodiments are herein described with reference to the accompanying drawings and more specifically FIGS. 1-13 and 15.

CSR is a form of periodic breathing believed to be due to instability in the central nervous system control of ventilation. Breathing in a CSR sufferer is characterized by a “waxing and waning” tidal volume as respiration alternates between repetitive episodes of apnea/hypopnea and hyperpnea. Recordings of nasal flow signals in a compressed time scale reveal a pattern that is similar to an Amplitude-Modulated ('AM') waveform.

During a period of Cheyne-Stokes breathing or CSR, the pattern of waxing and waning tidal volume that can be seen in a nasal flow signal as a direct measure of pulmonary function also is present as cyclical changes in other cardio-respiratory parameters such as blood oxygen saturation levels. For example, during sustained apneic periods, blood oxygen saturation may fall due to the dynamics of the cardio-respiratory system. Measurements of oxygen saturation using pulse oximetry exhibit periodic de-saturation and re-saturation that mimics the rise and fall of ventilation caused by CSR.

The cyclical pattern of the blood oxygen saturation levels in CSR differs to that of a serially occurring sequence of Obstructive Sleep Apnea (OSA) events. The patho-physiologic mechanism behind the Cheyne-Stokes breathing pattern is associated with the level of arterial partial pressures of carbon dioxide (PaCO₂). The presence of a low PaCO₂ may suppress patient's central drive to breathing in response to hypocapnia, which typically initiates shallow breathing and subsequently partial or complete withdrawal of breathing if driven below the apneic threshold, resulting in Central Sleep Apnea (CSA). Following an apneic period, a subsequent rise in PaCO₂ will develop, which may induce a hyper-ventilatory response. Consequently, a decline in PaCO₂ may begin where the cycle would normally repeat.

This oscillating response to ventilation may result in a waxing and waning tidal volume and a gradually swinging blood oxygen saturation levels. The rising and falling oxygen saturation levels are delayed but may usually be in synchrony with hyperventilation or hypoventilation. The underlying oscillation in the central respiratory drive in association with the cardiac and pulmonary interactions give rise to an oscillation in oximetric recording that are uniquely regular during CSR. The spectral feature is intended to capture this pattern of regularity in the oximetry signal as a marker of the CSR.

Evidence suggests that a compromised cardiac function is a risk factor to contributing to CSA. In the stable Congestive Heart Failure (CHF) population, prevalence rates of CSA ranging from 30% to 50% has been reported (Javaheri et al., Circulation. 1998;97:2154-2159.; Sin et al., Am J Respir Crit Care Med 1999;160:1101-1106.). It has also been supported that a high apneic threshold of PaCO₂ predisposes a development of CSA and CSR.

A period of pure Cheyne-Stokes breathing is commonly presented in a PSG study as a serially occurring sequence of CSA events. The development of CSA constituting pure Cheyne-Stokes breathing is non-hypercapnic in origin with typical cycle lengths of 60 seconds (Eckert et al., Chest, 2007; 131:595-607). It is to be differentiated from other forms such as idiopathic CSA or narcotic-induced CSA arising from the application of chronic pain medications. These forms of CSA typically have a much shorter cycle length. The selection of oximetric recordings used for the training of the classifier excludes such data as would be assessed and screened by the clinical expert during the pre-scoring process. This ensures only specific forms of CSA of interest are used for the training of the classifier.

CSR Versus OSA:

Cheyne-Stokes Respiration (CSR) is a form of periodic breathing that is typically observed through direct measurement of pulmonary functions such as a nasal flow recording or airway flow recording. Due to the coupling between the cardiac and pulmonary system, CSR may also be identified as alternating periods of desaturation and resaturation through an oximetry signal. Thus oximetry signals may provide a source of information available for the analysis of Cheyne-Stokes breathing. Benefits of this approach may include the use of oximeters for non-invasively measuring blood oxygen saturation levels, which is an important determinant of a subject's health condition. While oximetry recordings may provide evidence of the occurrence of CSR, or other breathing abnormalities which may also be reflected in an oximetry signal such as conditions of Obstructive Sleep Apnea (OSA). This is preferably taken into account during the training of the classifier to discriminate CSR from OSA.

OSA may be generally initiated by the collapse of the upper airway. During an OSA event, the central drive to breathing is not withdrawn as can be seen from the continuing respiratory effort during a PSG study. Initial breaths following an OSA event is typically deep in effort with large tidal volume, which is often associated with a rapid rise in oxygen saturation level. In a serially occurring sequence of OSA events, rapidly re-saturating oxygen saturation levels is thus believed to be indicative of an occurrence of OSA.

The occurrence of an OSA event is closely related to the mechanical state and anatomy of the upper airway. OSA is driven by the collapse of the pharynx, which may happen in a recurring manner but unlike CSR, it is not a form of periodic breathing. The variability in the length of time from the onset of a preceding OSA event to the onset of its successive OSA event tends to be shorter than the cycle lengths of CSR. Oximetry from an OSA recording may find a more episodic pattern of desaturation and re-saturation, lacking the typical regularity found in the cycle lengths of a pure CSR oximetry recording.

However, oximetry signals are contraindicated for use in diagnosing CSR by being prone to undesirable artifacts caused by body motion or limb movements. In adult recordings, oximeters are commonly placed at the fingertip or ear lobe. The quality of the oximetry signal is highly sensitive to any displacement of the optical sensor in an oximeter. Motion artifacts are typically characterized by periods in which abrupt de-saturation and sharp re-saturation occur. It is not uncommon to find that saturation levels are at zero percent within an artifactual period of oximetry recording. There may be a loss of information during this period, which may be unavoidable. This issue may be overcome by modifying the use of an oximetry signal to incorporate a detection scheme that takes into account the abruptness of de-saturation and re-saturation.

FIG. 1 depicts an example of an oximetry signal 102 and the derivative thereof or a derived oximetry signal 104 from a recording. The signal was recorded during CSR over the duration of a half hour (1800 seconds). Clear instances of artifacts are shown as the plunge to zero saturation and the sudden recovery. In a system or device of the present technology, data from the signals may be processed according to one or more of the following methodologies.

Identifying Artifacts

From the derived oximetry (SpO₂) signal 104 the beginning of an artifactual period where the signal goes from a negative value of less than −10% to a positive value of greater than 10% may be identified. The derived oximetry signal provides an indication of the beginning and end of an artifactual period, which is marked by an initial sharp negative spike followed by an abrupt positive spike. Artifacts may be removed by linearly interpolating across the region of artifacts.

Oximetry Signal Quality Indicator (QI)

Whereas oximetry measurements have been employed for the detection of OSA, those detection methods are not transferable to the problem of detecting CSR. The presence of CSR indicates central instability in ventilatory control. In pure Cheyne-Stokes breathing flow is often associated with central apneas and hypopneas. In contrast to obstructive apneas, the resumption of breathing in CSR is usually very gentle, which leads to a slower rate of re-saturation. The present technology takes into account this difference between OSA and CSR, by making use of the mean re-saturation period and the fact that our statistical analysis shows that only CSR demonstrates re-saturation longer than 10 seconds.

A a quality indicator may be defined for a derived oximetry (SpO₂) signal 104 by finding the number T of samples thereof where SpO2 drops below a predetermined percentage threshold such as 10%. The quality indicator (QI) may be defined as the ratio of T/N where N is the total number of samples considered. However, if this ratio is less than a threshold of, for example, about 0.75, the quality indicator may be set to zero. It is also possible to define the quality indicator as a function of the ratio T/N.

Calculation of an Event Feature

Once the artifacts have been identified they may be removed from the data. A signal of the remaining data may also be low-pass filtered to derive a filtered signal. The signal can be filtered first to remove unwanted and uninteresting high-frequency content. For example, the filter used may be a digital Finite Impulse Response (“FIR”) filter designed using the Fourier method with a rectangular window. In some embodiments, the filter may have a pass-band from 0 to 0.1 Hz, a transition band from 0.1 to 0.125 Hz and a stop band above 0.125 Hz. The number of terms in the filter varies with sampling frequency. The signal may be filtered by convolving the time series point-wise with a filter vector.

Next contiguous periods of re-saturation may be detected. The length of the period may be stored as components of a vector. The event feature may then be calculated as the mean of the components of the vector. The event feature can be associated with a quality indicator value. Thus, it may be output with a CSR determination based on the particular event feature to provide information to a clinician as to the quality of the CSR detection.

One alternative to extract an event from an oximetry signal may be to derive two filtered signals and then perform a comparison of their varying amplitudes to frame a desaturation event or resaturation event. The filter for the first of these derived signals shall have a very low cut-off frequency to represent the long-term saturation signal (SLong). The filter for the second of the derived signals may have a relatively higher cut-off frequency to represent the short-term saturation signal (SShort). When SShort falls below a threshold as a percentage of SLong, this may be taken as a trigger for recording the start of the desaturation event. When SShort subsequently rises above a threshold above SLong, this may be taken as a trigger to record as the end of the desaturation event. A similar process maybe applied to capture a resaturation event.

Calculation of a Spectral Feature (SF)

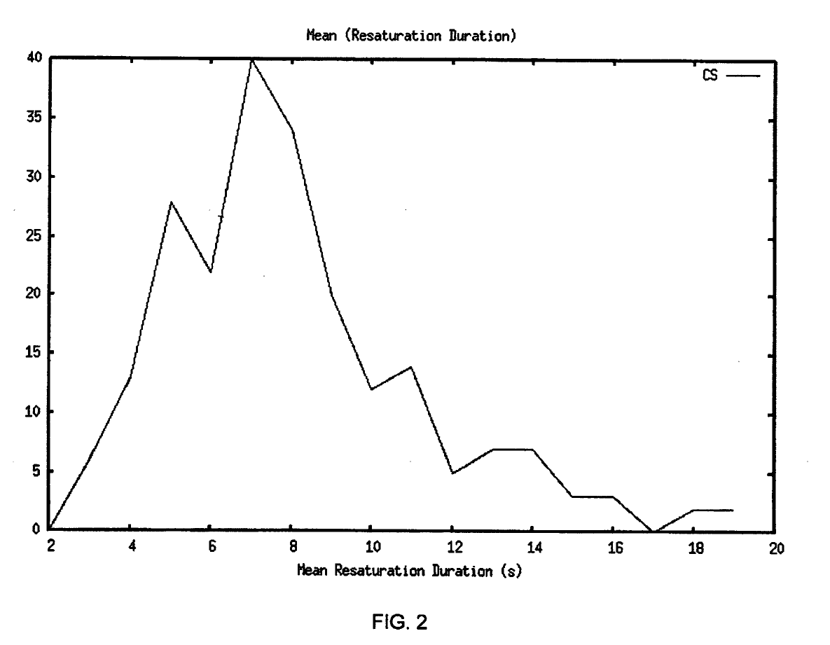

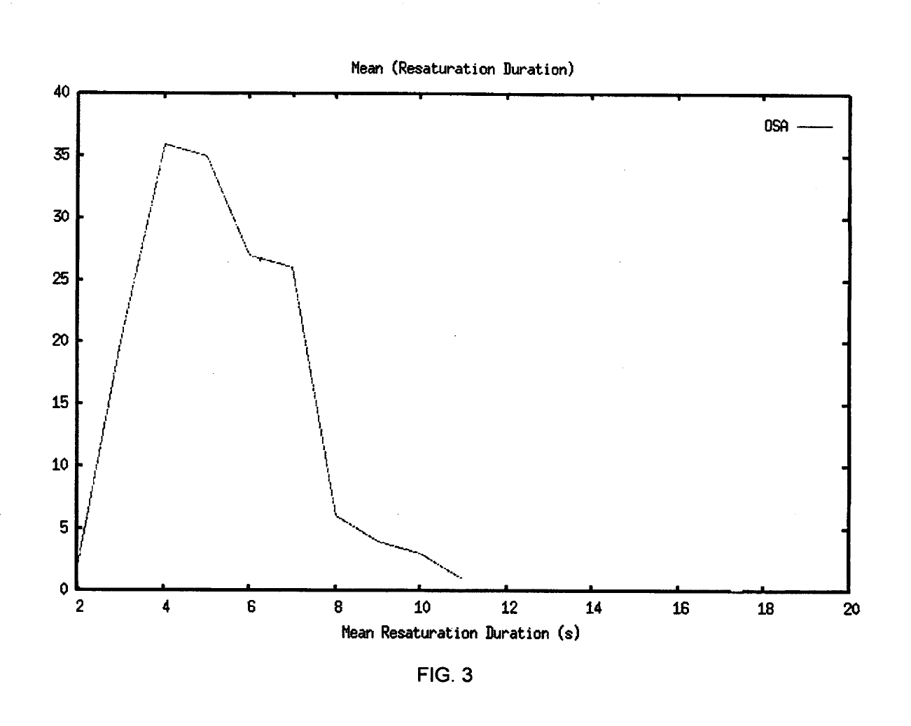

The periodic alternation between apnea/hypopnea and hyperpnea often leads to desaturation and resaturation that are delayed but in synchrony with breathing. The observed oscillation in SpO2 depends on multiple factors, one of which is the duration of an apnea. Longer apneas are associated with greater desaturation. FIGS. 2 & 3 show the distribution of mean saturation duration in CSR (FIG. 2) compared to those of OSA (FIG. 3) as a function of time measured in seconds. Observation of various CSR oximetry patterns finds a higher regularity, in contrast to the episodic nature of oximetry patterns during continuous periods of obstructive apneas. Using a Fourier transform, a spectral feature may measure the presence of a peak in the region near 0.083 Hz to 0.03 Hz.

The tendency to de-saturate and re-saturate over longer cycle times may be taken as a marker of a CSR abnormality. This may be detected or recognized using Fourier-transform techniques to determine individual frequency components and harmonics. Rapid resaturation during post-apneic termination of an OSA event with deep arousal breaths gives a more episodic style of desaturation and resaturation patterns. This distinguishes the frequency characteristics from the more regularly de-saturation and re-saturation patterns of CSR.

In some embodiments, some or all the following example steps may be implemented to determine a Spectral Feature using a Fourier-Transform analysis:

1. Remove artifacts

2. Divide the entire oximetry signal into discrete 30 minutes, 50% overlapping epochs

3. Subtract the signal from 100%

4. Subtract the resulting signal from an initial value and store this value

5. Low-pass filter the resulting signal

6. Add the initial value stored back to the filtered signal

7. Subtract the resulting signal from 100%

8. De-trend the signal by the mean value

9. Normalize the resulting signal using the Euclidean norm

10. Calculate the spectrogram with five half-overlapping epochs

11. Take the real and absolute magnitude of the spectrogram

12. Extract the 0.083-0.03 Hz region and form a new vector

13. The Spectral Feature (SF) is calculated as the difference between the maximum and the mean value

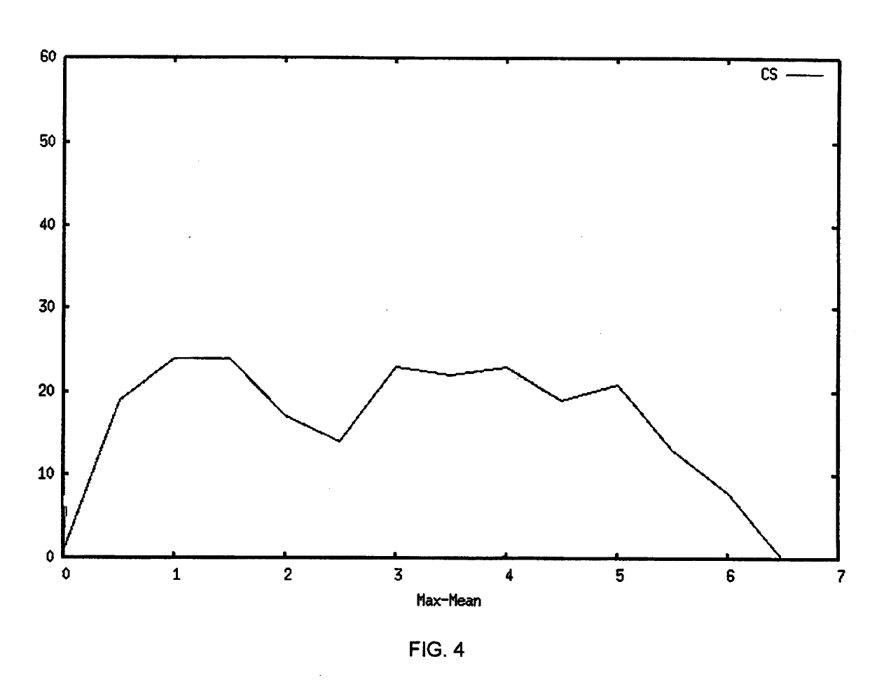

FIGS. 4 & 5 respectively depict the distribution of the spectral feature for CSR and OSA as the difference between the maximum and mean value of the Fourier-Transform as just described.

Use of Wavelet Transforms

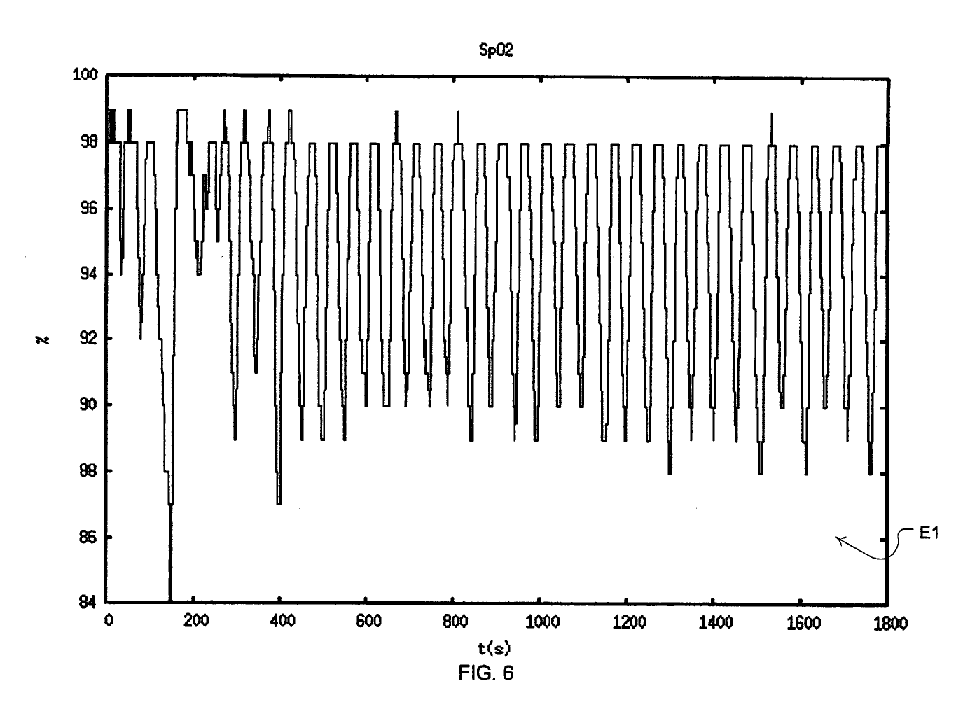

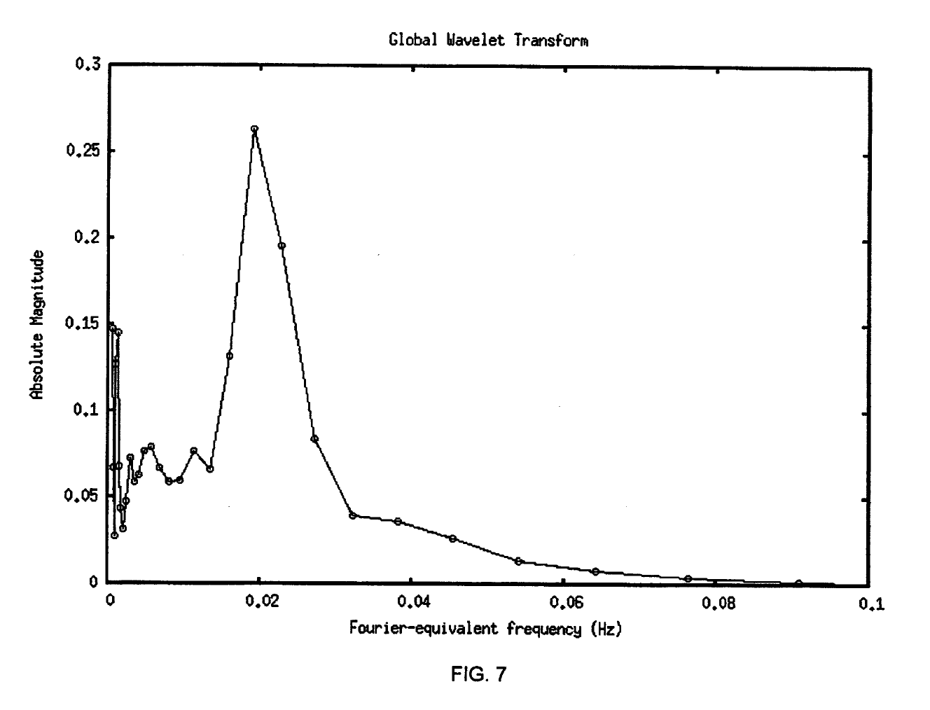

Continuous wavelet transform may also be applied to give time-frequency information over the duration of the signal. FIG. 6 shows the oxygen saturation with CSR occurring in a representative epoch E1In such CSR epochs, the wavelet-transformed data often results in a ridge that can be found or detected in the 2-dimensional data. The wavelet spectrum can be translated from the scale domain (dimensionless) into Fourier-equivalent frequency (Hz) depending on the type of wavelet transform used. FIG. 7 shows the global wavelet spectrum as a function of the Fourier-equivalent frequency using the Morlet wavelet as the wavelet function. Epochs with strong presence of CSR often find a spectral peak around the 0.02 Hz Fourier-equivalent region. This corresponds well with the Fourier-based spectral peak, as seen in FIG. 7. Thus, in some embodiments of the technology, the peak of the global wavelet spectrum may also be used as a spectral feature for the analysis of CSR in oximetry signal.

Delay of Saturation

The periodic alternation between apnea/hypopnea and hyperpnea often leads to desaturation and resaturation that are delayed but in synchrony with breathing. This Delay of the Saturation (“DoS”) level response is a result of the complex cardio-respiratory dynamics. Some or all of the steps of the following method may be used in some embodiments to extract the delay algorithmically.

1. Square the flow signal

2. Low-pass filter the squared flow signal

3. Square-root the resulting signal

4. Down-sample the signal to the equivalent frequency of the oximetry signal to give the ventilation signal

5. Normalize the ventilation signal by the absolute maximum value

6. Subtract the oximetry signal from 100%

7. Normalized by the absolute maximum value

8. Subtract the SpO2 signal from 1.0

9. Cross-correlate the normalized SpO2 signal with the down-sampled and normalized ventilation signal

10. Find the offset to the maximum cross-correlation attained

11. Calculate the delay in samples as the number of samples from the last index of the SpO₂ signal

12. Divide the delay in samples by the sampling rate to get the delay in seconds

Optionally, as an alternative to the aforementioned squaring and square root operations being performed on the flow signal in steps 1 and 3 above, an absolute value operation on the flow signal may be implemented.

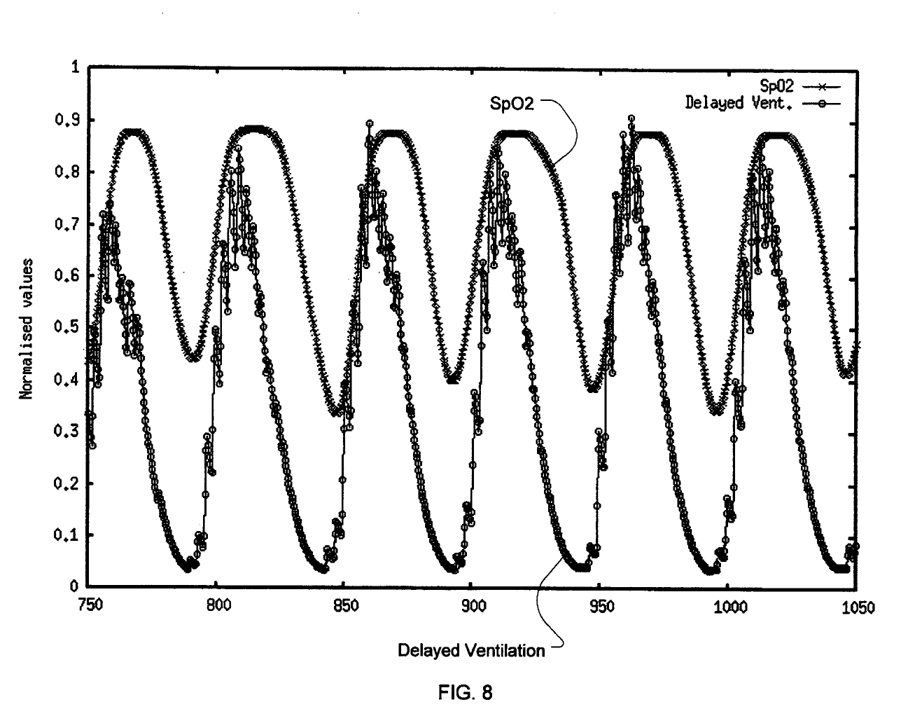

FIG. 8 shows a result of such a calculation by plotting a filtered SpO2 signal as a function of time in seconds and the shifted ventilation signal using the computed delay.

Training a Classifier to Discriminate CSR

The event feature and the Fourier-based spectral feature may be selected to train a classifier of the present technology. Training for an example embodiment was performed using 90 Embletta recordings of clinical diagnostic studies.

Two independent sets of polysomnographic (PSG) data were used for the development of the algorithm of a classifier. The first set (which is herein referred to as the EssenEmbla study) was a diagnostic clinical trial conducted at a sleep facility in Essen, North-Rhine Westphalia, Germany, involving 90 patients presenting with Central Sleep Apnea (CSA), OSA, and a combination of both. The EssenEmbla study was used as the training set. The second set (BadO) was conducted in Bad Oeynhausen, North-Rhine Westphalia, Germany. The prevalence of the BadO data set also contains recordings of CSA, OSA and a combination of both. These are 8 hours of overnight recordings that were then used as the test set to evaluate the classifier after a training session.

To facilitate the training of the algorithm of the classifier, initially both sets of data had been pre-classified by a clinician. Each of the recordings were scored by the resident clinical expert at ResMed in 30 minute segments, where a designation of predominant event is made by means of offline visual inspection through a computer with PSG software. The events were designated into one of five general types of events:

1. No apnea

2. CSR

3. OSA

4. Mixed apnea

5. Combination of events

As a result of this pre-classification process, each 8-hour recording yielded 16 non-overlapping epochs in total, each with a specified class of dominating event. In the EssenEmbla training set where 90 patients were involved, there was a total of 1440 classes of data available for training. Any residual epoch less than 30 minutes was not assessed. Nevertheless, the residual epoch may optionally be any period greater than several breath cycles of the patient. For example, the residual epoch may be greater than 5 minutes. The most preferred residual epoch may be 30 minutes.

During this pre-scoring process, the clinical expert utilized any of the available PSG channel recordings to assist in determining the predominant events and assigning a designation to each of the half-hour segment. These included the nasal flow, digital oximetry, measures of respiratory effort, sleeping position by means of gravitational indictors, heart rate, electroencephalography (EEG), electrocardiography (ECG), electromyography (EMG) and electro-oculography (EOG). Using the pre-classified designation of the training set, the oximetry and flow recording was segmented with a computer processor and software into strict 30 minute non-overlapping epochs of data for analysis. Selected epochs of specific pre-classified events were then used for exploring specific features to be used as indicators of CSR. By pre-classifying the data into half-hour epochs, the quantitative significance of particular short-term features was not diluted over the length of the entire recording.

The division of time for each epoch was based upon giving consideration to the typical occurrence and lengths of each CSR event. For a higher than average 90 seconds cycle length of waxing and waning pattern of CSR, assuming the oxygen saturation de-saturates and re-saturates at a similar pace, there are 20 continuous cycles of CSR that can be captured within half an hour, which was sufficient for analysis. According to the American Academy of Sleep Medicine (AASM) 1999 published guidelines for standards of PSG diagnostic criteria, mild obstructive sleep apnea (OSA) defined as where on average between 5 to 15 events per hour of greater than 10 seconds cessation of breathing is found in a recording. In a 30 minute epoch with the presence of mild OSA, there will be at minimum 2.5 events within half an hour.

The decision boundary was formed using a Bayesian classification technique. This method is appropriate for normally distributed data and aims to find a discriminate that optimally separates the two classes (CSR and non-CSR) with minimum risks. Other classification methods may also be used to derive the decision boundary. Such examples may include neural networks or the k-nearest neighbor scheme.

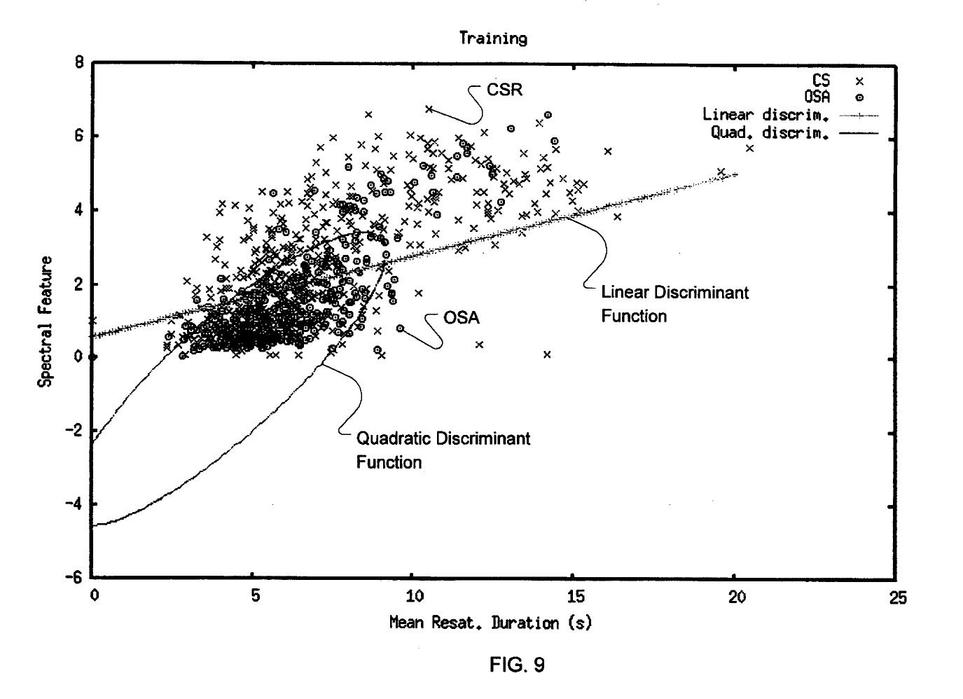

FIG. 9 illustrates the decision boundary and its relationship to the distribution of the data after training on an epoch-by-epoch basis. The straight line represents the linear discriminant function and the elliptical line represents the quadratic discriminant function following Bayesian classification. The discriminant function divides the space into regions of CSR and non-CSR.

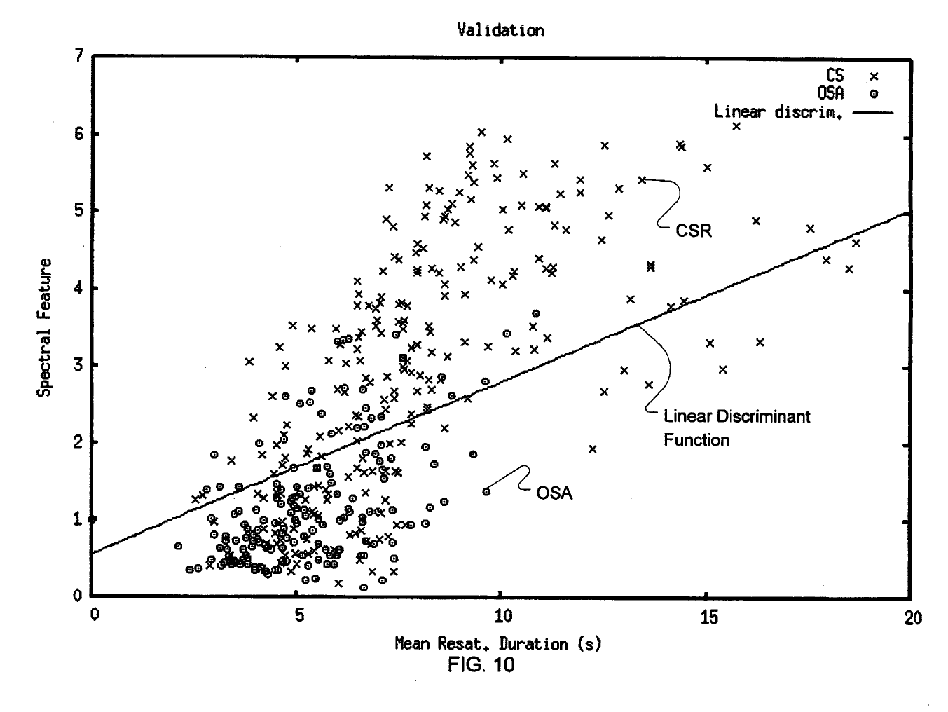

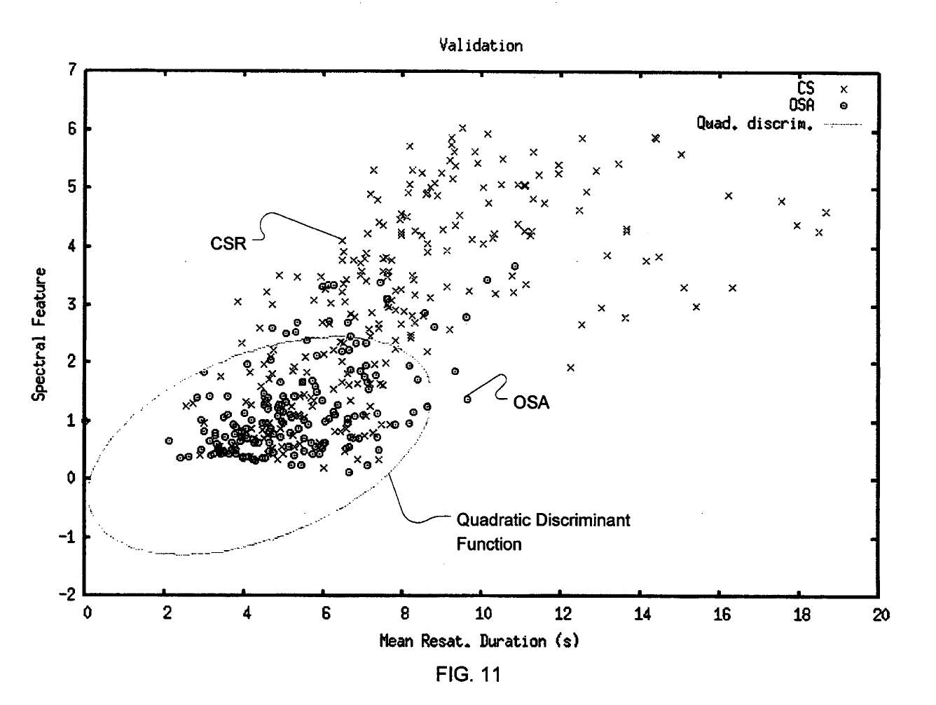

FIGS. 10 and 11 illustrate the trained decision boundary applied to the validation test data set on an epoch-by-epoch basis. The overall probability for the entire SpO2 recording may be derived using the following series of steps.

1. Calculate the probability by mapping the perpendicular distance to the decision boundary using the sigmoid function

$$p = \frac{{a}^{d}}{1 + {a}^{d}}$$

2. If the probability is greater than a specified threshold such as 0.5, then the epoch will be classified as the CSR.

3. If any of the epochs are classified as CSR, the oximetric recording will be classified as CSR-probable

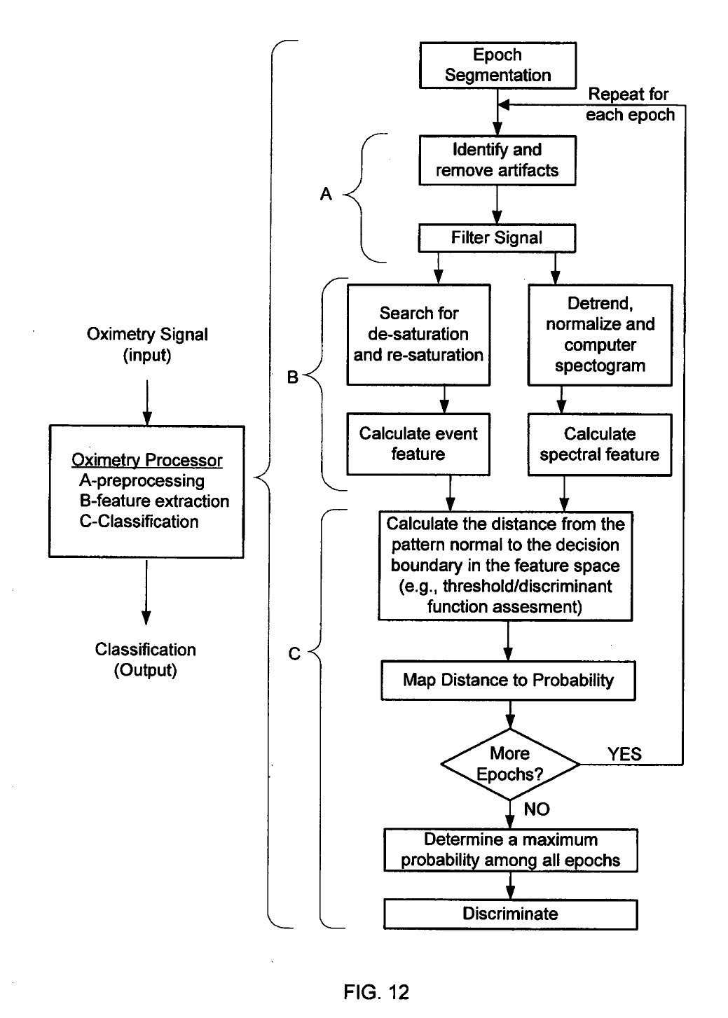

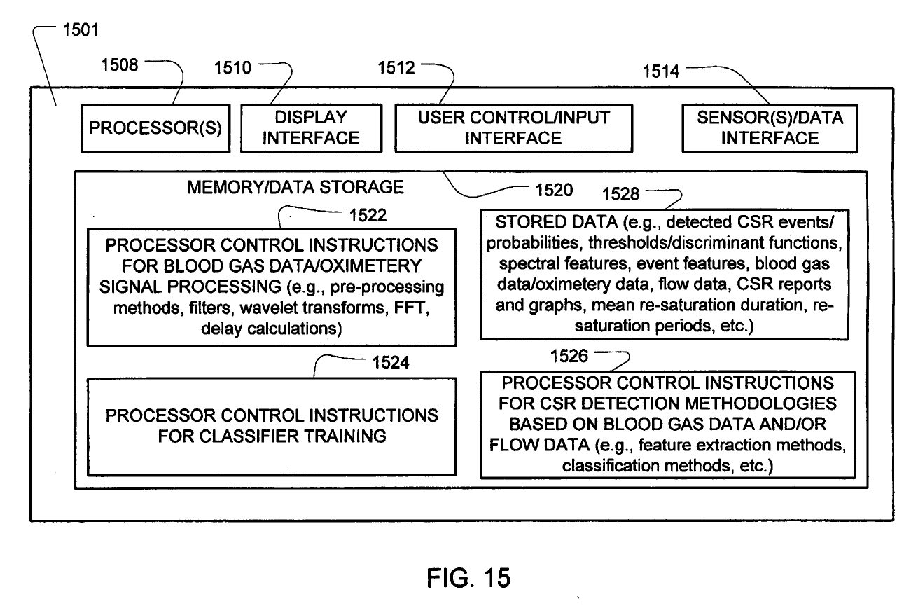

FIG. 12 is a flow chart of example steps just described for feature extraction and classification. Such a methodology may be implemented as software or in the circuits or memory of a detection device as further illustrated in FIG. 15.

Patient-by-Patient Classification and Results

Probability Values

To get an understanding of how well the classifier discriminates CSR on a patient-by-patient basis, although it may be implemented to do so, instead of simply determining a binary output (CSR or non-CSR) for each epoch, the classifier may be implemented to produce a probability value of between zero and one for each epoch segment. For each derived mean resaturation duration and spectral feature, calculate the distance normal from the data point in the feature space to the decision boundary. This perpendicular distance is then mapped to a probability value where the probability is a function of the distance from the decision line.

$$p = \frac{{e}^{d}}{1 + {e}^{d}}$$

If the distance is zero i.e. (d=0) the feature value would coincide with the boundary, then the probability is exactly 0.5. As the distance increases to positive infinity, the probability asymptotically tends towards 1.0. As the distance increases to negative infinity, the probability asymptotically tend towards 0.0. By defining the region of feature space corresponding to CSR as positive distance from the discriminant in this embodiment, CSR may be defined as any resulting probability value of greater than 0.5. It will be recognized that the technology may be implemented to yield other values for distinguishing the presence of CSR with distance from such a discriminant function.

In the process of classifying an oximetry recording on a patient-by-patient basis, a processor implemented algorithm embodying the classifier may be programmed to iterate through the entire length of the signal, calculating a probability value for each half hour epoch, where the window increments by half an epoch per iteration (i.e. quarter of an hour). The iteration proceeds until all half-hour epochs have been processed and a vector of probability values for the recording can be obtained.

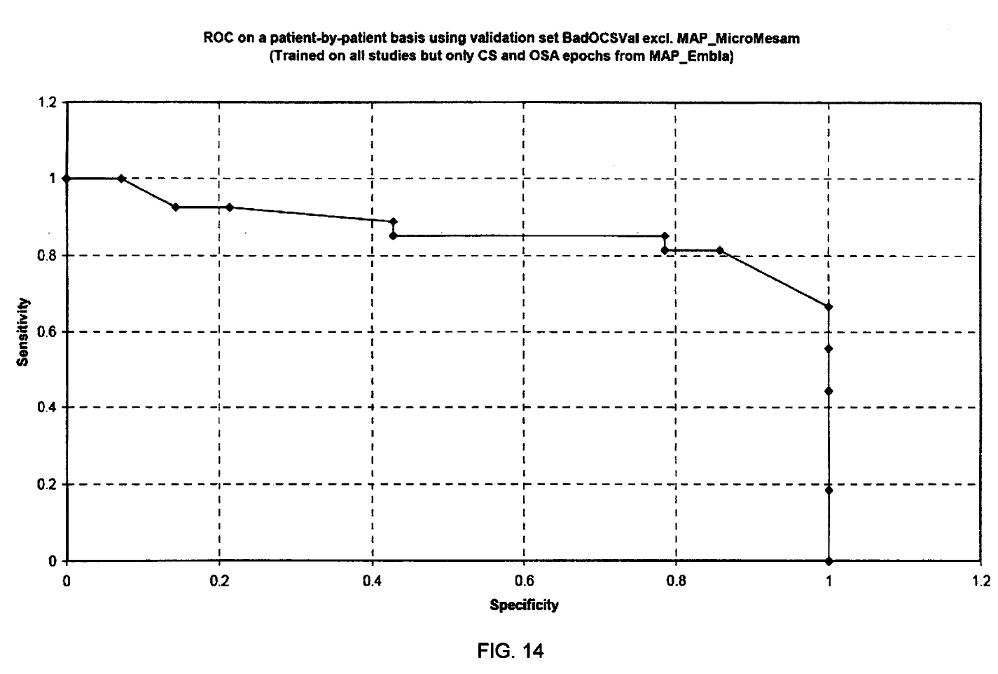

The overall probability of CS for a single patient/recording may be calculated using the maximum probability found for all epochs classified. The overall performance of the classifier then may be evaluated over the testing set by incorporating a threshold for the decision of CS. This may yield receiver-operating characteristics (ROC) such as the example depicted in FIG. 14.

Each point in FIG. 14 on the ROC curve represents a 0.05 increment/decrement of probability over its adjacent point. The maximum area is achieved at a threshold probability of 0.75 when sensitivity is 0.8148 and specificity is 0.8571. By raising the threshold probability further to 0.8, full specificity can be achieved at the expense of a lower sensitivity of 0.6667. The following table summarizes the key performance measures on a patient-by-patient basis:

| | |
|---|---|
| Threshold chosen (based on max area) | 0.75 |
| Sensitivity | 0.814815 |
| Specificity | 0.857143 |
| Prior probability assumed | 0.004 |
| Positive Predictive Value (PPV) | 0.02069 |
| Negative Predictive Value (NPV) | 0.99883 |
| False Alarm Rate (FAR) | 0.97931 |
| False Reassurance Rate (FRR) | 0.00117 |
| Positive Likelihood Ratio (LR+) | 5.703704 |
| Negative Likelihood Ratio (LR−) | 0.216049 |

Note that this table assumes a prior probability of 0.004 for patients with CS. This estimate is based on a prevalence of 0.01 of Americans with Congestive Heart Failure (CHF) whose age is over 65 years old reported in the Sleep Medicine Reviews (2006) 10, 33-47 by Jean-Louis Pepin et al. Within the CHF population, a prevalence of one-third to one-half is commonly reported in literature on CSR. By taking the prevalence value for CS within the CHF population as 0.4, the prior probability is calculated as 0.01 multiplied by 0.4, which equals 0.004.

The positive likelihood ratio (LR+) indicates that if a patient is classified as CS positive overall, the pre-test probability of that patient truly having CS is boosted by a factor of 5.7 times. Similarly, the negative likelihood (LR−) if a patient is classified as CS negative overall, the pre-test probability of that patient actually having CS is lowered by a factor of 0.22. LR+ and LR− together indicate to the clinician, the strength of a diagnostic test. According to the rating on the qualitative strength of a diagnostic test by Dan Mayer in his book Essential Evidence-Based Medicine, an LR+ and LR− of 6 and 0.2 respectively is considered “very good”. Thus, the diagnostic performance of the example classifier on a patient-by-patient basis can be considered close to “very good”.

Application

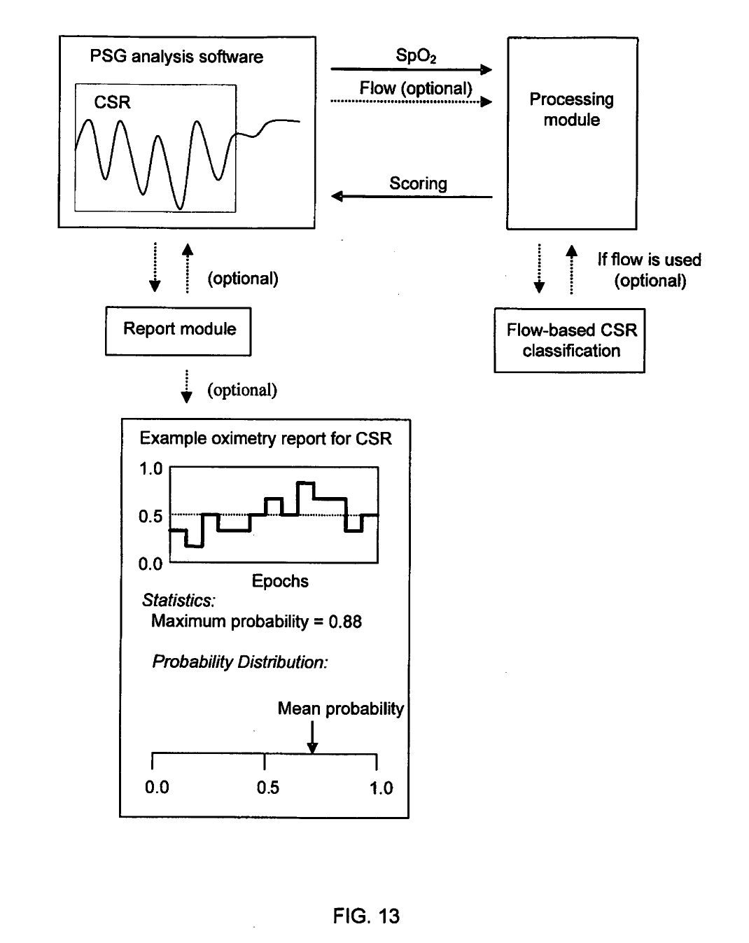

One application of such a classifier when implemented by a programmed processor or other processing device is to enable clinicians to screen a large number of patients for evidence of CSR as a computer-aided diagnostic tool. One instance of such application may be used in the environment of home sleep testing, wherein a sleep physician issues a portable SDB screening device such as ApneaLink™ with an oximeter to a patient. Preferably, sleep data may be collected overnight for subsequent analysis by the physician. This analysis by the physician or clinician may be performed offline, that is, after the use of the measuring device in one or more sleep sessions. For example, an algorithm embodying the classifier can be implemented as a module for sleep study analysis software such as Somnologica™ (manufactured by a company called Embla) or ApneaLink™ (manufactured by ResMed Limited). This may allow the automatic scoring of CSR to be marked on an oximetry signal trace or graph. An example embodiment is illustrated in the schematic of FIG. 13. A complementary feature would be a module that automatically generates a report based on the classification results computed by the algorithm. Clinicians would then be able to use the report as a summary to support their decision-making process. Optionally, such a classifier algorithm may be implemented within an SDB screening device to generate data on a display message having a classification of CS as previously discussed.

Furthermore, in some embodiments, the aforementioned oximetry classifier of the present technology may be used or implemented in conjunction with a flow rate classifier, such as the flow rate classifier disclosed in U.S. Patent App. Pub. No. 20080177195, the entire disclosure of which is incorporated herein by reference. For example, in such an embodiment, a controller with one or more programmed processors may include both an oximetry classifier algorithm and a flow rate classifier algorithm. The flow rate classifier may detect the delivered or measured flow rates and then analyze the flow rates with determinant functions and then classify the flow rates based on threshold amounts. A CS probability indicator generated by the controller may then be based on both classifier algorithms, for example, by combining the probability data from each, by using a scheme such as one based on the average of both probabilities or the maximum of either probability as the final conclusion drawn from both classifiers. Such a controller may have increased accuracy and generally better results.

Accordingly, embodiments of the present technology may include a device or apparatus having one or more processors to implement particular CSR detection and/or training methodologies such as the classifiers, thresholds, functions and/or algorithms described in more detail herein. Thus, the device or apparatus may include integrated chips, a memory and/or other control instruction, data or information storage medium. For example, programmed instructions encompassing such detection and/or training methodologies may be coded on integrated chips in the memory of the device or apparatus. Such instructions may also or alternatively be loaded as software or firmware using an appropriate data storage medium. With such a controller or processor, the device can be used for processing data from an oximetry signal. Thus, the processor may control the assessment of a CSR occurrence or probability as described in the embodiments discussed in more detail herein. Moreover, in some embodiments, the device or apparatus itself may optionally be implemented with an oximeter or other blood gas measurement device to measure blood gas itself and then implement the methodologies discussed herein. In some embodiments, the processor control instructions may be contained in a computer readable recording medium as software for use by a general purpose computer so that the general purpose computer may serve as a specific purpose computer according to any of the methodologies discussed herein upon loading the software into the general purpose computer.

An example embodiment is illustrated in FIG. 15. In the illustration, the CSR detection device 1501 or general purpose computer may include one or more processors 1508. The device may also include a display interface 1510 to output CS detection reports, results or graphs as described herein such as on a monitor or LCD panel. A user control/input interface 1512, for example, for a keyboard, mouse etc. may also be provided to activate the methodologies described herein. The device may also include a sensor or data interface 1514 for receiving data such as programming instructions, oximetry data, flow data, etc. The device may also typically include a memory/data storage components. These may include processor control instructions for blood gas data/oximetry signal processing (e.g., re-processing methods, filters, wavelet transforms, FFT, delay calculations) at 1522 as discussed in more detail herein. They may also include processor control instructions for classifier training methodologies at 1524. They may also include processor control instructions for CSR detection methodologies based on blood gas data and/or flow data (e.g., feature extraction methods, classification methods, etc.) at 1526. Finally, they may also include stored data 1528 for these methodologies such as detected CSR events/probabilities, thresholds/discriminant functions, spectral features, event features, blood gas data/oximetery data, flow data, CSR reports, mean resaturation duration, resaturation periods, etc.

While the technology has been described in connection with what are presently considered to be practical and preferred embodiments, it is to be understood that the technology is not to be limited to the disclosed embodiments, but on the contrary, is intended to cover various modifications and equivalent arrangements included within the spirit and scope of the technology.

1. A method for indicating the presence of Cheyne-Stokes respiration from blood oxygen saturation levels measured by an oximetry signal comprising: identifying and removing artifactual oximetry periods from the oximetry signal to produce a second signal; and with a processor, determining a mean length of contiguous periods of resaturation in the second signal and generating a positive indication of Cheyne-Stokes respiration based on an extent of the determined mean length.

2. The method of claim 1 wherein the positive indication is generated when the extent of the determined mean length is greater than a predetermined threshold.

3. The method of claim 1, further comprising filtering the second signal to remove high frequencies.

4. The method of claim 3, further comprising performing frequency analysis on the second signal to determine an extent of oscillation in the oxygen saturation level, and wherein a positive indication of Cheyne-Stokes respiration is generated for oscillations over longer cycle times.

5. The method of claim 4, wherein the second signal is Fourier analyzed to determine the extent of oscillation in the oxygen saturation level and wherein a positive indication of Cheyne-Stokes respiration is generated for peaks in the Fourier-based spectrum at about 0.02 Hz.

6. The method of claim 4, wherein the second signal is wavelet analyzed to determine the extent of oscillation in the oxygen saturation level and wherein a positive indication of Cheyne-Stokes respiration is generated for oscillations over longer cycle times.

7. A method for indicating the presence of Cheyne-Stokes respiration from blood oxygen saturation levels measured by an oximetry signal and a ventilation flow signal comprising:
determining with a processor a delay of blood oxygen saturation level data compared to ventilation flow level data having either an apnea or hypopnea and an hyperpnoea; and
generating a positive indication of Cheyne-Stokes respiration for determined delays above a predetermined threshold.

8. A method for training a classifier to discriminate Cheyne-Stokes respiration from blood oxygen saturation levels measured by an oximetry signal comprising:
pre-classifying polysomnographic data to obtain non-overlapping epochs each with a specified class of dominating event;
with a processor, segmenting oximetry and flow recordings into non-overlapping epochs of data having a predetermined length of time; and
forming a decision boundary with a processor to discriminate Cheyne-Stokes respiration and non-Cheyne-Stokes respiration classes of events with the epochs.

9. The method of claim 8 wherein the predetermined length of time is greater than five minutes.

10. The method of claim 8, wherein the predetermined length of time is approximately 30 minutes.

11. The method of claim 8 further comprising:
determining distance from the decision boundary for each event and normalizing the distance to a probability value for each epoch.

12. The method of claim 11, wherein the predetermined length of time is approximately 30 minutes.

13. The method of claim 12, wherein the equal length epochs are as long as the recordings.

14. The method of claim 13, wherein the epoch length is approximately the length of a representative hypopnoea-hyperpnoea sequence.

15. A device for detecting the presence of Cheyne-Stokes respiration from an oximetry signal and a ventilation flow signal, wherein said device identifies and removes artifactal oximetry periods from the oximetry signal to produce an second signal; and wherein the device determines the mean length of contiguous periods of re-saturation in the second signal and returns a positive indication of Cheyne-Stokes respiration, if said mean length is greater than a predetermined threshold.

16. The device of claim 15, wherein the device filters high frequencies from the second signal.

17. The device of claim 15, wherein said oximetry signal is compared to a first set of threshold values by a first classifier, and said ventilation flow signal is compared to a second set of threshold values by a second classifier.

18. A computer implemented method of detecting an occurrence of Cheyne-Stokes respiration with one or more programmed processors comprising:
accessing blood gas data representing a measured blood gas signal;
determining a duration of one or more contiguous periods of changing saturation of a blood gas from the blood gas data;
detecting the occurrence of Cheyne-Stokes respiration from a comparison of the determined duration and a threshold derived to differentiate saturation changes due to Cheyne-Stokes respiration and saturation changes due to obstructive sleep apnea.

19. The method of claim 18 wherein the one or more contiguous periods of changing saturation comprises re-saturation periods and the measured blood gas signal comprises an oximetry signal.

20. The method of claim 19 wherein the determined duration comprises a mean period length and wherein the detecting indicates an occurrence when the mean period length exceeds the threshold.

21. The method of claim 20 wherein the threshold comprises a discriminant function.

22. The method of claim 21 wherein the detecting the occurrence further comprises determining a distance from the threshold and comparing the distance to a further threshold.

23. The method of claim 21 further comprising determining a presence of a peak in a predetermined frequency range for de-saturation and re-saturation cycles of the blood gas data and comparing the determined presence to the discriminant function.

24. The method of claim 23 further comprising processing the blood gas data to remove artifact data.

25. The method of claim 24 further comprising measuring the blood gas with an oximeter.

26. An apparatus to detect an occurrence of Cheyne-Stokes breathing, the apparatus comprising:
a memory for blood gas data representing a measured blood gas signal;
a processor coupled with the memory, the processor being configured (a) to determine a duration of one or more contiguous periods of changing saturation of a blood gas from the blood gas data and (b) to detect an occurrence of Cheyne-Stokes respiration from a comparison of the determined duration and a threshold derived to differentiate saturation changes due to Cheyne-Stokes respiration and saturation changes due to obstructive sleep apnea.

27. The apparatus of claim 26 wherein the one or more contiguous periods of changing saturation comprises re-saturation periods and the measured blood gas signal comprises an oximetery signal.

28. The apparatus of claim 27 wherein the determined duration comprises a mean period length and wherein the detecting indicates an occurrence when the mean period length exceeds the threshold.

29. The apparatus of claim 28 wherein the threshold comprises a discriminant function.

30. The apparatus of claim 29 wherein the processor is configured to detect the occurrence by further determining a distance from the discriminant function and comparing the distance to a further threshold.

31. The apparatus of claim 29 wherein the processor is further configured to determine a presence of a peak in a predetermined frequency range for de-saturation and re-saturation cycles of the blood gas data and comparing the determined presence to the discriminant function.

32. The apparatus of claim 31 wherein the processor is further configured to process the blood gas data to remove artifact data.

33. The apparatus of claim 32 further comprising an oximeter, coupled with the processor, to generate the blood gas signal.

34. An apparatus for indicating the presence of Cheyne-Stokes respiration from blood oxygen saturation levels measured by an oximetry signal comprising:
means for identifying and removing artifactual oximetry periods from the oximetry signal to produce a second signal; and
means for determining a mean length of contiguous periods of re-saturation in the second signal and generating a positive indication of Cheyne-Stokes respiration based on an extent of the determined mean length.

35. An apparatus for indicating the presence of Cheyne-Stokes respiration from blood oxygen saturation levels measured by an oximetry signal and a ventilation flow signal comprising:
means for determining a delay of blood oxygen saturation level data compared to ventilation flow level data having either an apnea or hypopnea and an hyperpnoea; and
means for generating a positive indication of Cheyne-Stokes respiration for determined delays above a predetermined threshold.

36. An apparatus for detecting an occurrence of Cheyne-Stokes respiration comprising:
means for accessing blood gas data representing a measured blood gas signal;
means for determining a duration of one or more contiguous periods of changing saturation of a blood gas from the blood gas data;
means for detecting the occurrence of Cheyne-Stokes respiration from a comparison of the determined duration and a threshold derived to differentiate saturation changes due to Cheyne-Stokes respiration and saturation changes due to obstructive sleep apnea.
